(19) United States
(12) Patent Application Publication
(10) Pub. No.: US 2024/0365832 A1
(43) Pub. Date: Nov. 7, 2024

(54) METHOD FOR PROCESSING A STARCH HYDROLYSATE AND STARCH HYDROLYSATE

(71) Applicant: food´or International GmbH, Köln (DE)

(72) Inventors: Karl LUDOVICI, Köln (DE); Sebastian HANFT, Köln (DE); Thomas HÄSSLER, Köln (DE); Timo KOCH, Elsdorf (DE); Birgit KAUFMANN, Köln (DE)

(73) Assignee: food´or International GmbH, Köln (DE)

(21) Appl. No.: 18/578,375

(22) PCT Filed: Jul. 12, 2022
(86) PCT No.: PCT/EP2022/069509

§ 371 Date: Jan. 11, 2024

(30) Foreign Application Priority Data
Jul. 12, 2021 (DE) 10 2021 117 932.7
Jan. 21, 2022 (DE) 10 2022 101 408.8

(51) Int. Cl. A23L 29/30; A23J 1/00; A23J 1/14; A23J 3/20; A23L 11/00; A23L 11/50; C12P 19/02; C12P 19/12; C12P 19/14; C12P 21/06

(52) U.S. Cl. CPC A23L 29/35; A23J 1/008; A23J 1/14; A23J 1/148; A23J 3/20; A23L 11/05; A23L 11/50; C12P 19/02; C12P 19/12; C12P 19/14; C12P 21/06

(57) ABSTRACT

The invention relates to a method for processing a starch hydrolysate, in which at least one legume species is provided. This is separated into a first fraction and a second fraction by a sift grinding process of the provided at least one legume species. Thereby, the first fraction comprises a higher protein content than the second fraction. In the second fraction, a proportion of the starch contained in the provided at least one legume species is at least 40% by weight. The second fraction is processed to produce a starch hydrolysate which, after hydrolysis, comprises a protein content in the range of 5% to 30% by weight.

### CROSS-REFERENCE TO RELATED APPLICATION

The present application is a national stage of International Patent Application No. PCT/EP2022/069509 filed on Jul. 12, 2022, which claims priority to German applications DE 10 2021 117 932.7 of Jul. 12, 2021 and DE 10 2022 101 408.8 of Jan. 21, 2022, the disclosure content of which is hereby incorporated by reference in its entirety. The invention relates to a method for processing a starch hydrolysate, a starch hydrolysate and a fungal protein mixture. The invention also relates to a process for the production of lactic acid.

### BACKGROUND

The use of protein-containing raw materials, especially on the basis of legumes, for the production of vegetarian or vegan food products has recently increased significantly. Not only conventional approaches based on soy are being pursued, but legumes, such as pea and fava bean, are also increasingly being used. However, pulses have a relatively large starch content, which is removed during processing of the pulses to extract the protein content. For this purpose, a so-called wet extraction or a so-called dry extraction can be used, among others. In wet extraction, the separation between the starch portion and the protein portion is carried out by means of an aqueous solution, whereby the proteins are precipitated by suitable adjustment of a pH value and separated from the remaining starch and fibers. Subsequently, the starch can again be dried and further used. In the case of dry extraction, processing is carried out by means of so-called sift grinding, in which the legume is ground very finely and then separated into a starch-containing and a protein-containing fraction, respectively, by means of a classifier screen.

An increasing difficulty in the processing of pulses and other fruits for the production of proteins is the relatively high starch content that remains after extraction of the protein content. Although this starch fraction is currently processed in various other processes, the demand for starch or carbohydrates for agriculture, cosmetics and cattle feed production is covered. As a result, the expected increase in processing of pulses will produce additional starch for which there is currently no market, so that this could lead to a further drop in the already low price of starch. There is thus a risk that the processing of pulses will become economically unviable. Accordingly, there is a need to further process the by-products, in particular starch-containing by-products, in order to increase the total yield in the value chain when processing pulses.

### SUMMARY OF THE INVENTION

This need is met by the objects of the independent patent claims. Designs and further aspects are the subject of the subclaims.

The inventors have recognized that the starch-rich fraction, as a by-product of dry extraction of pulses, has a not inconsiderable protein content in addition to the starch content. During further processing of this fraction, in particular processing of the starch, either the protein fraction remains or, depending on the application, is also consumed. According to the proposed principle, a method for processing starch is now to be created in which this protein fraction continues to be used, so that the result is an intermediate product suitable for further processing, which comprises a processed starch fraction on the one hand and a still unprocessed protein fraction from the pulses on the other.

To this end, the inventors propose a method for producing a hydrolysate with increased protein content and for processing a starch hydrolysate. In this process, at least one type of legume is first provided, which is then ground by a sift grinding process and separated into a first fraction and a second fraction. The first fraction comprises a higher protein content than the second fraction and is referred to as the high-protein fraction. In contrast, the second fraction, whose protein content is lower, comprises a higher starch content and is also referred to as the starch-rich fraction.

According to the proposed principle, the starch-containing fraction thus includes at least 40% by weight, but in particular at least 50% by weight, of the starch contained in the legume provided. In addition, the second fraction may also include fiber constituents and other coarser residues from the sift grinding process. The second starch-containing fraction, which also comprises a protein content in the range of 5 to 30% by weight, is now hydrolyzed to produce a starch hydrolysate. This creates a starch hydrolysate that comprises a protein fraction that is still unprocessed and is a residue from the sift grinding process of the at least one legume species.

This is based on the knowledge that in a sift grinding process the protein-containing fraction, i.e. the first fraction, comprises a significantly smaller particle size than the second starch-containing fraction. Consequently, components that exceed a certain particle size remain in the starch-containing fraction. This fraction may also contain fats or other components of the legume.

The starch hydrolysate from the starch-rich fraction can now be further processed in various ways using microorganisms. The term microorganisms is to be broadly defined here and includes all unicellular, but also less cellular organisms. Microorganisms include bacteria, yeasts, fungi, fungal mycelia, algae and protozoa. The various microorganisms include the respective wild types, but also types that have been genetically modified or modified by other methods.

Generally, the starchy fraction and the carbohydrates contained therein are fermented to subsequently produce the desired target product(s). Such fermentation can be both anaerobic and aerobic, depending on the desired target product. In addition to the production of proteins, components of proteins in the form of biomass (baker's yeast, vitamins or amino acids such as lysine, methionine and threonine or oils and fats), this also includes the production of organic acids, in particular lactic acid, succinic acid, itaconic acid or acetic acid, to name just a few. Alcohols can also be produced, as well as diols, in particular propanediol and butanediol. In addition, medical starting materials and/or products can also be produced, such as insulin, hyaluronic acid, streptokinase and a variety of antibiotics (e.g. penicillin).

In addition to the above examples, in some aspects the starch hydrolysate with the additional substances also forms starting materials for biodegradable plastics. Finally, for the food sector, the hydrolysate can also be used for the production of proteins via fermentation with fungal mycelium.

The non-sugar components still present in the starch-containing reaction, such as the remaining vegetable proteins, but also minerals, oils and fats, can be metabolized by the microorganisms or remain in the end product. Of course, the protein-containing components can also be removed before processing, but the use of the non-sugar-containing components has the advantage that they do not have to be added during fermentation, or only in part. This reduces the cost of producing higher-value substances from the hydrolysate.

In the following, among others, both the further processing by means of a fungal mycelium and the fermentation with microorganisms for the production of lactic acid are explained in more detail. It is understood here that the end substances in these two specific examples, namely a protein mixture and lactic acid, respectively, can be replaced by the end products mentioned above. In this case, parameters have to be adjusted depending on the desired end product, but the starting material, namely the hydrolysate produced here from a fraction obtained from a sift grinding process and containing starch but still comprising a protein mixture, is the same in each case. In this respect, the intermediate product produced by sift grinding and subsequent hydrolysis can serve as a starting material for a variety of other industrially valuable products.

According to some aspects of the proposed principle, the starch hydrolysate thus obtained can be cultivated using a fungal mycelium from the division of the Basidiomycota, the Ascomycota and/or also Fusarium species with the starch hydrolysate as well as an additional nitrogen source. An additional nitrogen source is useful because in this case the fungal mycelium obtains the nitrogen necessary for growth from the additional nitrogen source and does not have to rely on the protein components still present in the hydrolysate for this purpose. After cultivation, the result is dried and ground to produce a fungal protein mixture. Alternatively, it can also be processed directly without additional drying. In addition to a fungal protein component, the fungal protein mixture also comprises residual components of proteins of the legume species. With the method proposed in this way, the processing of legume protein can thus be scaled up so that a protein mixture can also be obtained from the starch-containing component of the legume species in the end result.

By using a sift grinding and a dry extraction process, the processing costs are significantly reduced compared to a wet extraction process. At the same time, by hydrolyzing the coarse second fraction and subsequent cultivation with, for example, a microorganism, the remaining protein fraction present in the second fraction is further utilized in a cost-effective and very efficient manner. The proposed process can thus be used to produce a protein mixture from a legume species with a very high specific weight fraction in a cost-effective manner. The two fractions produced in this way are, on the one hand, a protein mixture with a high proportion of the legume protein and, on the other hand, a second end product which, depending on the further processing, can comprise a residual proportion of legume protein.

In a further embodiment of the proposed process, the first fraction may be further processed to produce a protein isolate having a legume protein content in the range of 80% by weight to 97% by weight, and in particular in the range of 85% by weight to 95% by weight. For this purpose, the first fraction can be subjected to a wet extraction process so that remaining smaller starch particles and other substances are removed from the mixture during the wet extraction process, thus enriching the protein content.

In one aspect, the hydrolyzing step comprises additional filtering, in particular membrane filtering and or precipitation of the second fraction. This removes coarser fibers and other fiber-containing portions in this fraction. This may expediently contribute to the production of a protein isolate or concentrate with a very high protein content. Some further aspects deal with the possibility of exploiting, after hydrolysis and saccharification, the different sizes of the resulting sugar and the other proteins and fats present from the starch-rich fraction. To this end, in some aspects, it is envisaged to separate protein fractions and/or fat fractions from the hydrolysate, so as to still obtain a highly enriched protein-containing and/or fat-containing fraction. The remaining sugar mass can be used for fermentation. In addition to various filtration methods, mechanical methods such as decantation, centrifugation or other mechanical methods can be used for separation.

In a further aspect, the process comprises dehulling the legumes prior to the step of classifying. In addition, the second fraction can also be additionally screened prior to the hydrolyzing step so that residual materials with a particle size greater than 100 μm, in particular greater than 60 μm to 70 μm, are removed from the second fraction. This ensures that, above all, only starch components and residual protein components as well as fats and minerals remain in the starch-containing fraction, but no more fiber components.

The second starchy fraction, which is hydrolyzed, includes a fat fraction in addition to a residual protein fraction. This is originally part of the legume and in some aspects may be greater than 0.5% by weight, particularly in the range of 1% to 6% by weight. It should be noted in this regard that degreasing may be performed in upstream processing steps or from as part of the sift grinding process, such that the fat content contained in the first or second fraction may shift or be adjusted in both amount and composition. In some aspects, the fat present in the legume is milled primarily into the first fraction and thus into the proteinaceous fraction. In other aspects, the fat content in the starch-rich fraction is increased by suitable means. In some aspects, the breakdown of the fats present in the legume species is also not uniform, but the distribution of the different fatty acids varies depending on the fraction.

Some aspects concern the composition of the starchy fraction. Thus, experiments on several examples have shown that the starchy fraction comprises in the range of 50% by weight to 70% by weight of the total mass, and in particular 55% by weight to 65% by weight. The starchy fraction may also be in the range of 60% by weight to 67% by weight, in the range of 55% by weight to 63% by weight, or even in the range of 57% by weight to 64% by weight. However, as mentioned, proteins and fats are still included. In some aspects, the contents of proteins are in the range of 10% by weight to 35% by weight, but more particularly around 20% by weight to 25% by weight of the total mass, for various starch-containing fractions of legumes obtained according to the present method prior to hydrolysis. Similarly, in some examples, the amount of legume protein is in the range of 22% by weight to 27% by weight, or 18% by weight to 23% by weight. Although there are also concentrations of 12% to 20% by weight, these require particularly fine or even multiple sifting, depending on the application.

Overall, however, the amount of protein in the starch-containing fraction is greater than 15% by weight and more particularly greater than 20% by weight and more particularly greater than 22% by weight or even greater than 24% by weight but less than 30% by weight. In some examples, the amount of starch and proteins was about 76% by weight to 90% by weight, and in particular the amount is between 79% by weight and 85% by weight, with the remainder of the total mass consisting of water, fats and ash. The fat content is essentially between 0.8% by weight and 1.6% by weight, with values between 1% by weight and 1.4% by weight often occurring. However, in some examples, values greater than 1.5% by weight or even greater than 2.25% by weight are possible. Thus, in some aspects, the fat content can range from 0.5% by weight to 5.0% by weight, but more particularly can range from 1.0% by weight to 4.5% by weight, or can range from 1.5% by weight to 4% by weight, or can range from 1.0% by weight to 3.5% by weight.

Ash, i.e., minerals and residual constituents, in some aspects ranges from 1.0% by weight to 8.0% by weight and, in particular, ranges from 1.5% by weight to 6.5% by weight. In other aspects, the ash ranges from 2.0% by weight to 3.9% by weight. In some examples, the amount of ash is greater than 1.5% by weight, or greater than 2.0% by weight, or greater than 2.5% by weight, or greater than 3.0% by weight, or greater than 3.5% by weight, or greater than 4.0% by weight, or greater than 4.5% by weight, but still less than 8.0% by weight. This showed that individual percentages of starch, proteins and fats, and ash were within the ranges indicated but without any particular correlation between them.

In other words, even with the same type of legume used, for example fava bean, but different arable soils on which the legume is grown and/or from which the legume is harvested, there appear to be slight differences in the ratios even with otherwise identical classifier milling. Conversely, slightly different sift grinding was found to result in a different composition. Thus, longer sift grinding and separation at smaller particle size appears to result in higher starch concentration at the expense of protein quantity.

Of course, different legume species show different proportions in the respective components, but most of them can be characterized by a combination of the indicated ranges. In this respect, therefore, any combination of the indicated ranges, sub-ranges or even individual values therefrom can be combined with each other without this being generally detrimental to the subsequent process. On the contrary, depending on a later use, a larger fat content or protein content may be expedient to increase the biological value of the sifted base substance but also of the hydrolysate.

Some further aspects of the process deal with the step of hydrolyzing or generally converting the starch portion of the starch-containing fraction into a sugar mixture. In particular, the step of hydrolyzing may be carried out enzymatically, using an enzyme selected from a group consisting of the enzymes mentioned further below.

In an alternative aspect, hydrolysis is carried out with an acid, with neutralization of the acid, in particular with a basic nitrogen compound, following completion of the hydrolysis. In this aspect, an ammonium-containing salt is formed, which can also serve as a nutrient for subsequent cultivation with a microorganism. In an alternative aspect, neutralization of the mixture is performed as well as a subsequent shift of the pH to the basic range with a basic nitrogen compound, which forms a nitrogen source for later processing, for example for cultivation with a microorganism, in particular a fungal mycelium or for submerged fermentation.

The hydrolysate obtained in this way comprises not only the various sugars, which can be adjusted either by acid or enzymatically depending on the hydrolysis, but also the residual protein fraction in the range from 5% to 35% by weight. This fraction can be separated mechanically as described above, i.e. by filtering, decanting or the like, so that an additional side stream of highly enriched protein is formed.

Often, the hydrolysate comprises a sugar mixture of different sugars, such as glucose, fructose, maltose, sucrose and other oligo- and polysaccharides in different weight proportions. In this regard, the production of the individual sugar types as well as their weight fraction is adjusted by the use of the corresponding enzymes or via the process parameters. In one aspect, the various sugars are selected to be particularly suitable for subsequent cultivation with the aforementioned microorganisms. In some aspects, an amylase is used that operates primarily at lower temperatures. This has the advantage that the temperature-dependent components recognized with advantage above, in particular vitamins, for example but not limited to the B complex, folic acid, and/or proteins from the starting material are largely retained and do not denature or decompose. Such a process is thus particularly useful for the mixtures from the sift grinding process according to the principle proposed here.

This accelerates the process speed, especially in a downstream cultivation process with microorganisms.

The legume species used can be a single legume species, but also a mixture of these. Possible legume species include in particular soybeans, peas, green or white beans, fava beans, chickpeas, peanuts, lentils, lupin, and combinations thereof

Another aspect relates to a starch hydrolysate which comprises a sugar content of at least 40% by weight, but in particular at least 50% by weight and in particular greater than 60% by weight. The sugar content comprises at least one of the following sugars, namely glucose, fructose, maltose and sucrose, said sugar content being represented by a proportion of at least 10% by weight. According to the proposed principle, the starch hydrolysate further comprises a legume protein mixture, in particular from pea or fava bean, with a proportion of less than 30% by weight.

In some aspects, glucose is primarily present, and in some aspects ranges from 60% by weight to 96% by weight of the sugars present. In some aspects, glucose may also be present up to 98% by weight of the sugars. In particular, glucose and other monosaccharides are present in some aspects at greater than 80% by weight of the total sugars present. In addition, oligosaccharides or components thereof may also be present in the hydrolysate that are remnants of the sifting process and have not been converted or not fully converted. In one aspect, the legume protein mixture may comprise a proportion in the range of 5% to 30% by weight in the hydrolysate. In an enzymatic saccharification, the protein content is in the ranges already mentioned in the base material. In some aspects, it may be particularly around 20% to 25% by weight of the total mass. After hydrolysis, the protein content may be greater than 15% by weight and more particularly greater than 20% by weight and more particularly greater than 22% by weight or even greater than 24% by weight but less than 30% by weight of the total mass. In some aspects, the proportion may be slightly higher after hydrolysis than before.

In some examples, the proportion of proteins and/or amino acids is somewhat higher than the values originally present in the base material. The reason for this is enzymatic saccharification, which breaks down further proteins in some of the remaining residual components, so that these contribute to the total protein amount in the hydrolysate.

Thus, the amount of protein in a hydrolysate is in the range of 10% by weight to 35% by weight, but more particularly around 15% by weight to 25% by weight of the total mass. Similarly, in some examples, the amount of protein is in the range of 18% by weight to 23% by weight, or 21% by weight to 27% by weight, or even between 7.5% by weight and 20% by weight. In some aspects, the amount of protein or amino acids is 0.10% by weight to 0.65% by weight higher than the corresponding value of the base substance.

The fat content in the hydrolysate is essentially between 0.8% by weight and 1.6% by weight, with values between 1% by weight and 1.4% by weight occurring in most cases. However, in some examples, values greater than 1.5% by weight or even greater than 2.25% by weight are included. Thus, in some aspects, the fat content can range from 0.5% by weight to 5.0% by weight, but more particularly can range from 1.0% by weight to 4.5% by weight, or can range from 1.5% by weight to 4% by weight, or can range from 1.0% by weight to 3.5% by weight.

The individual fatty acids or fat components as well as their amount depend on the legume type, which also contributes to the protein mixture. It was also surprisingly found that the proportion of mono-unsaturated and poly-unsaturated fatty acids is in the range of more than 70% by weight of the total amount of fat present, and often even more than 80% by weight of the total amount of fat, with the proportion of polyunsaturated fatty acids predominating and in itself already accounting for more than 55% of the total fat content in the starch hydrolysate. The proportion of saturated fatty acids, on the other hand, is lower and amounts to less or approximately the same as the amount of monounsaturated fatty acids.

The inventors have recognized that a starch hydrolysate prepared by the method disclosed above comprises a relatively high proportion of B vitamins in the hydrolysate as well, due to the presence of B vitamins in the original legume. In one aspect, therefore, a proportion of B vitamins of more than 0.002% by weight of the starch hydrolysate is disclosed

Surprisingly, it was found that the vitamin B content does not decrease even through hydrolysis. The complex is thus also present in the starch hydrolysate and can thus be actively used for further processing of the hydrolysate. This can be particularly useful if the vitamin B complexes are growth factors of microbiological components or fungi to which the starch hydrolysate is added.

In some aspects, the amount of B vitamins present is about 1.5 mg to 6 mg based on 100 g total mass. In other aspects, the amount of vitamin B complexes present can range from 1.8 mg to 5.6 mg or can range from 2.0 mg to 5.1 mg per 100 g total mass. In further aspects, the amount of vitamin B complexes is above 2.2 mg per 100 g of total mass and can be, for example, between 2.5 mg and 4.7 mg or even between 2.8 mg and 4.2 mg per 100 g of total mass. A large proportion above 50% may be accounted for by vitamin B3. Possible components of the vitamin B complex in the starch hydrolysate are thiamine, niacin, pantothenic acid and pyridoxine, pyridoxal and pyridoxamine.

In addition, it was found that various amino acids are also present in the hydrolysate as part of the proteins and proteins. Besides aspartic acid in the range of 2% by weight to 3.5% by weight, in particular between 2.2% by weight to 3.3% by weight and especially between 2.5% by weight to 3.0% by weight, these are also glutamic acid in the range of 3% by weight to 5% by weight resp. between 3.5% by weight to 4.3% by weight or also in the range of 3.6% by weight to 4.4% by weight, and arginine in the range of 1.6% by weight to 2.6% by weight and in particular between 1.9 and 2.2% by weight. Overall, in some aspects, the above, as well as lysine and valine, may have a percentage above 1% by weight. In further aspects, the amino acids proline and glycine and at least one of isoleucine, serine, alanine and phenylalanine are present in the range of 0.8% by weight to 1.2% by weight and in particular between 0.9% by weight and 1.1% by weight. In contrast, the proportion of threonine and tyrosine, as well as hystidine, in fava bean is less than 1% by weight and often less than 0.85% by weight. The proportions of the amino acids taurine, hydroxy-proline, hydroxy-lysine and g-aminobutyric acid, on the other hand, are below 0.2% by weight or even below 0.1% by weight.

In addition, a starch hydrolysate produced by the process may further comprise other components or dietary fiber and, generally speaking, non-proteinaceous components having a particular particle size from the preceding sift grinding process. In some aspects, the non-proteinaceous components exhibit a particle size in the range of 30 μm to 120 μm with a maximum in the range of 40 μm to 100 μm.

Another aspect deals with the further use of the starch hydrolysate either with the protein portion still present or after its separation. In some aspects, the hydrolysate is fermented. In this regard, the starch hydrolysate can be used, for example, as a nutrient for bacteria, yeasts, algae, and/or fungi. Examples of fermentation would include: Alcohols such as bioethanol, or organic acids, citric acid and acetic acid. Fungi can be used to ferment the hydrolysate to form amino acids or proteins. In some aspects, the starch hydrolysate is added to yeasts.

An exemplary further processing of the above-mentioned hydrolysate would be the production of lactate, such as L-lactate but also enantiomerically pure D-lactate, since the latter can be further processed into biodegradable plastics, so-called polyactides. In fact, the demand for lactic acid has been growing for some years, so that this is a possible source of further processing of the hydrolysate. For the production of lactic acid it is possible to use lactic acid bacteria but also fungi or algae. Because of their high lactate production and low by-product formation, some representatives of the genera Lactobacillus, Leuconostoc, Pediococcus, Carnobacterium, Lactococcus, Streptococcus, Enterococcus, Vagococcus, Aerococcus, Alloiococcus, Oenococcus, Sporolactobacillus, Tetragenococcus, and Weissella are particularly suitable. Depending on the strain, they are capable of producing L- or D-lactate in high enantiomeric excess.

Fungi of the genus Rhizopus, especially Rhizopus oryzae, are suitable for the production of L-lactate with high enantiomeric purity. Furthermore, these fungi also thrive with fewer nutrients and lower pH values.

Lactic acid bacteria require certain amino acids and vitamins depending on the strain. The use of a low-purified hydrolysate, i.e. with a continued high residual protein content, has therefore proved advantageous because the further addition of nutrients can be reduced, thus lowering the cost of lactic acid production. Such a mixture with a high protein content can also be referred to as a protein hydrolysate, i.e., a complex mixture of peptides of different chain length as well as free amino acids. The latter may already be present in the mixture after a sift grinding process but may also be generated from the existing proteins by suitable measures, for example by the addition of proteases.

Possible further utilization and thus cost reduction similarly applies to trace elements left over from the sifting and hydrolysis process, which have growth-promoting effects depending on the strain used. Thus, in some aspects, lactic acid bacteria of at least one of the above strains are mixed with the hydrolysate in an aqueous solution. In this regard, the pH and temperature of the solution may be adapted to the needs of the strain used. In some aspects, these are bacteria of at least one of Sporolactobacillus laevolacticus, Sporolactobacillus inulinus, Sporolactobacillus putidus, Lactobacillus lactis, Lactobacillus delbrueckii and subtypes thereof, Lactobacillus coryniformis and subtypes thereof, and Leuconostoc mesenteroides. Different species can also be combined, for example to take advantage of the fact that some species require different amino acids or can synthesize certain amino acids required by other species.

In some aspects, additional amino acids are added, particularly when the proteins and amino acids remaining in the starch-containing fraction are insufficient for biosynthesis, thereby inhibiting lactate production. In other aspects, the proteins present are digested prior to feeding the bacteria, thereby increasing the amount of free amino acids in the hydrolysate. In this respect, a protein and starch hydrolysate are ultimately provided and used for lactic acid production. Incidentally, such a protein and starch hydrolysate can also be used for the further uses and refinements of the starch-rich fraction described herein, i.e., for fungal protein production described further below or for the production of alcohols and the like. Digestion of the proteins from the starch-rich fraction can be carried out enzymatically during hydrolysis, but also afterwards or before.

Another aspect deals with a fungal protein mixture comprising a first protein portion from a fungus from the division of the Basidiomycota or the Ascomycota, and a second protein portion. The second protein portion is such that a lysine portion or an arginine portion in the entire fungal mycelium is increased relative to the lysine portion or the arginine portion, respectively, in the first protein portion. In other words, the second protein portion comprises a lysine portion or arginine portion that complements the portions in the first protein portion. Thus, in some aspects, the second protein portion is formed at least in part from the protein portion that remains in the starch-rich fraction during the sift grinding process according to the proposed principle, which in turn serves as the basis for producing the hydrolysate used for fungal myceliation.

In this way, a fungal mycelium or fungal protein mixture is created that increases the naturally low levels of lysine and arginine in a pure fungal protein mixture.

In one aspect, this second protein portion comprises a legume protein, particularly a pea protein, a fava bean protein, or a combination thereof.

### DESCRIPTION OF THE DRAWINGS

Further aspects and embodiments according to the proposed principle will become apparent with reference to the various embodiments and examples described in detail in connection with the accompanying drawings.

FIG. 1 shows a first embodiment of a method for the production of a protein isolate as part of a processing of pulses

FIG. 2 shows an example of a process flow for a dry insulation process according to some aspects of the proposed principle;

FIG. 3 illustrates an embodiment of a process according to the proposed principle;

FIG. 4 shows another embodiment of a process for the production of a fungal mycelial protein or fermentation for the production of lactic acid according to some aspects of the proposed principle;

FIG. 5 shows a distribution of particle size of a dry isolation process with sift grinding to separate the two fractions according to some aspects of the proposed principle.

### DETAILED DESCRIPTION

The following embodiments and examples show various aspects and their combinations according to the proposed principle. It is understood that the individual aspects and features of the embodiments and examples shown in the figures can be readily combined with each other without affecting the principle of the invention. Some aspects are indicated in ranges. It should be noted that minor deviations from these may occur in practice, but without contradicting the inventive idea.

For the purpose of this application, the term “plant protein” includes a plant protein mixture. Such a plant protein mixture is obtained from a plant species in the manufacturing process. This may be, for example, fava bean or pea or another legume.

Such legumes have a protein content, starch, and other components such as fibers, minerals, fats, vitamins and others. Various processes are used for processing and, in particular, for extraction or separation of the protein and starch components, which will be explained in more detail below. However, the result is plant protein isolates and plant protein concentrates, each of which describes plant protein mixtures that are present in different concentrations. The other components of an isolate or concentrate can come from the range of fats, sugars including starch, cellulose, fibers and water. The concentration of the protein mixture in the respective isolate or concentrate depends not only on the type of processing, but also on the process steps within each process, resulting in a variety of mixtures with different concentrations and residual components.

For example, a plant protein isolate is a mixture of a plant protein in which the concentration of the protein mixture is in the range above 83% by weight, for example in the range from 87% to 97% by weight. For a protein concentrate, the weight fraction is in the range below 80%, for example in the range of 40% to 75% to about 80%.

Unless otherwise stated, a “plant protein” includes a plant protein mixture from the respective plant, otherwise it is referred to as a “single plant protein”.

In a corresponding manner, a “pea protein” or a pea-based plant protein is a protein mixture that comprises essentially pea, pea components from the pea plant and has been processed. Accordingly, a legume-based protein is a protein that has been derived from legumes. Similarly, a fava bean protein comprises such a mixture based on fava bean.

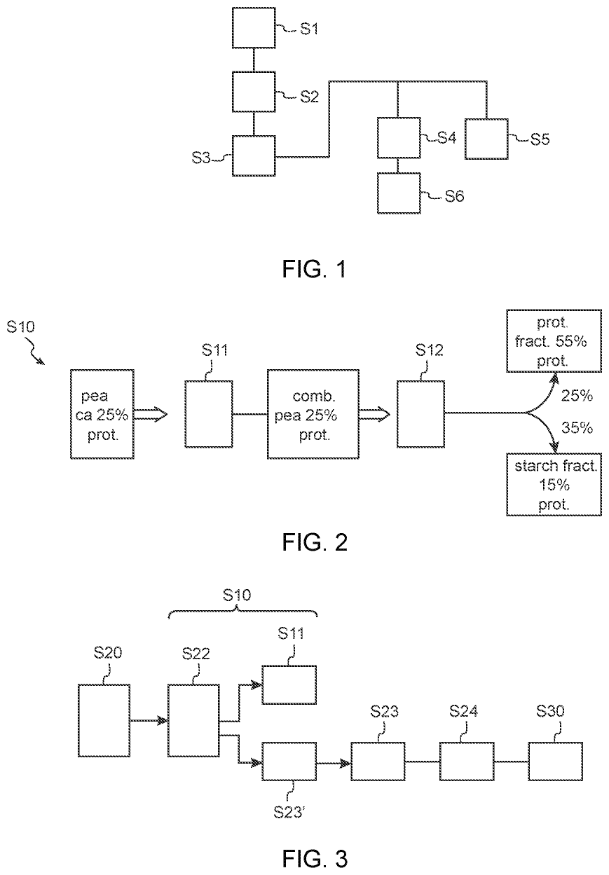

FIG. 1 shows an overview of a conventional wet extraction process with its main process steps. In simplified terms, a type of legume is separated into its main components, namely a protein-rich fraction and a starch-rich fraction, so that these can be processed separately. The protein content of the legume is essentially in the range of 25% by weight. The remaining 75% by weight is divided among starch, fat, fiber and dietary fiber, minerals, vitamins and other substances. Likewise, the legume continues to include a not insignificant water content.

In a first step S1, the legume is separated from its shell in a suitable mill and the two components are separated from each other. Thus, the actual legume is then present as such without its shell. In a second step S2, the legume is ground and dissolved in water. This produces a liquid interspersed with proteins, starch and sugars, as well as other substances. In step S3, the proteins are precipitated by adding various chemicals to shift the pH. These settle to the bottom of the solution due to the chemicals added. Through various extraction and separation processes, the fraction is enriched with proteins and separated from the rest of the solution. Subsequently, the two fractions further processed are essentially separately, with chemical neutralization in the protein-rich fraction being the first step in step S4. The protein-rich fraction thus contained is dewatered and dried in several steps via different processes.

The second fraction, which mainly contains starch, is further processed in various ways in step S5. In addition to possible filtering to separate fibers and other substances, the starch-containing fraction can also be washed again, dewatered and then dried. The wet extraction process recorded here allows the protein components of the legume to be almost completely separated from the remaining components and enriched to very high concentrations. In this way, a protein isolate comprising a very high concentration fraction of pure legume protein is produced in step S6. Depending on the processing effort, the starch-containing fraction comprises only a residual protein content in the range of a few % by weight of the total second fraction.

However, the process described here is costly both in terms of investment and in terms of energy consumption due to the various extraction and drying processes, especially for the production of protein isolates with very high concentrations of a protein mixture. It has been found that, due to the high starch content, this process is only profitable under certain conditions. The background to this is the rather low price on the market for the starch obtained, since starch also occurs in cereals and other products as a main or side stream, and the quantity available on the market sometimes exceeds the demand or, in general, the market appears saturated. The starch must therefore be further processed.

A simplified wet extraction process with fewer complex extraction steps reduces expenses considerably, but results in a starchy fraction in which the protein content is higher. The proteinaceous fraction is thus less concentrated and in turn produces lower proceeds, partially offsetting the benefit from reduced costs. The inventors now propose to further process a starchy fraction with a higher protein content using the concept according to the invention described below, so that a very good cost-benefit ratio is nevertheless achieved overall.

A process different from the wet extraction process is shown in FIG. 2 in the form of a dry extraction process or a so-called protein shift. This forms part of the process according to the proposed principle. In this process, a legume species, for example pea, fava bean, chickpea or others, is provided in step S10 and its shell is removed in a subsequent process similar to the wet extraction process.

After removal of the shell, the legume is subjected to a fine grinding process in step S11. This process grinds the legume significantly finer than is the case with the usual grinding process during a wet extraction. The legume ground in this way is then subjected to a classifier separation in step S12, which results in a so-called protein shift. This makes use of the fact that, as a result of the preceding fine grinding process, the various components of the legume comprise a distribution with regard to their particle size. In particular, protein components have a somewhat smaller particle size than the corresponding starch-containing components or the starch itself. Fats, minerals and the other elements are divided between the two fractions, although various process parameters can easily shift them in one direction or the other.

This aspect is illustrated in FIG. 5 by the diagram there, which shows the two main fractions with their respective particle size distribution. The first proteinaceous fraction has a grain size distribution in the range of essentially less than 10 μm and is represented by curve K1. In contrast, the starch-containing fraction represented by curve K2 has a particle size distribution whose maximum is in the range of 50 μm to 100 μm and thus significantly exceeds the average particle size of the protein-containing fraction. The two fractions are separated from each other by the sifting separation in step S12 downstream of the process in step S11 of FIG. 2. The separation takes place, for example, along a predefined particle size and is shown in FIG. 5 by the dashed line, for example in the range of 20 μm.

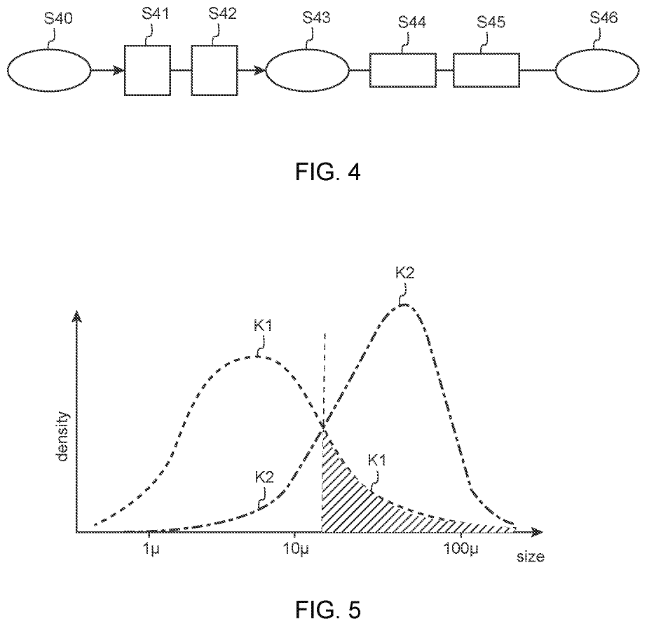

Constituents smaller than 20 μm thus fall into the proteinaceous fraction, the total proportion of which is in the range of 25% by weight of the total. The protein content is 55% to 60% by weight within this protein-containing fraction. Components with a particle size above 20 μm form the starch-containing fraction, whereby smaller amounts of protein as well as fibers and others are also added here, since these also comprise a larger particle size. The residual protein content is in the range of 10% to 15% by weight based on the amount of this fraction. This is illustrated in FIG. 4 by the two curves K1 and K2. The area of curve K1 with a particle size greater than 20 μm is significantly smaller than the other area, but nevertheless falls into the starch-containing fraction and is thus lost to the protein-containing fraction. Fibers and other parts have a significantly larger particle size. Only a small portion of starch with very small particle sizes remains in the proteinaceous fraction.

In contrast to the wet extraction process, the dry extraction process of FIG. 2 thus does not yield a protein isolate with a protein concentration of more than 75% by weight, but only a concentrate in the protein-containing fraction with approx. 50% to 65% by weight of protein. By appropriate grinding in steps S11 and sifting at a previously defined particle size, the protein content in the starch-containing fraction and the protein-containing fraction can be shifted slightly, but the protein content is still somewhat lower as a result compared with the wet extraction process. The advantages of the dry extraction process, however, are significantly lower investment and operating costs, which compensates for the somewhat poorer yield compared with the wet extraction process. In addition, the starch-containing fraction can be further refined with the concept proposed by the inventors, thus significantly increasing the value of the starch-containing fraction.

In this context, as already mentioned, starch is now a major component of production in the processing of cereals and pulses, so that the value of starch or carbohydrates from starch is relatively low. Although this is somewhat offset by the dry extraction process used and its lower operating costs, the inventors have nevertheless set themselves the goal of further processing the starch-containing fraction in a suitable manner in both the wet extraction process and the dry extraction process, and of increasing its value again through the downstream process steps.

In this regard, FIG. 3 shows an embodiment of the proposed process for producing a starch hydrolysate. In this, a legume species is provided in step S20. This comprises, for example, pea, fava bean, lentil, green bean, chickpea, peanuts, lupine, soybeans or combinations thereof. In step S22, these are first freed from the shell, ground and then sifted.

This results, as already explained in the previous embodiment example of FIG. 2, in a protein-containing fraction with a protein content in the range of 50% to 65% by weight, and a starch-containing fraction with a residual protein content in the range of 15% by weight measured against the starch-containing fraction. The starch-containing fraction is used in step S23′ as a starting material for further processing to produce a starch hydrolysate. In a first step S23, the starch-containing fraction is screened to remove the coarser components above a certain particle size, e.g. above 100 μm particle size. These are mainly components such as fibers and dietary fiber, i.e., the non-starchy or proteinaceous components of the fraction. The mixture screened in this way thus comprises a proportion of starch in the range of at least 40% by weight, but in particular at least 50% by weight to 65% by weight.

The following table compares, by way of example, the starting material of a starch-containing fraction of a legume used as a starting material for hydrolization according to the proposed process with a starch isolate of another legume. Since several series of experiments were performed with several sifting operations, either the respective averages are indicated or ranges are indicated, especially for amino acid values. The ranges are also included earlier in this disclosure.

| | | Legume fraction | Starch isolate |
|---|---|---|---|
| Dry matter | [g/100 g] | 90.2 | 89.2 |
| Water | [g/100 g] | 9.8 | 10.8 |
| Fat | [g/100 g] | 1.3 | <0.5 |
| Total protein | [g/100 g] | 23.0 | <0.625 |
| Ash | [g/100 g] | 2.8 | <0.1 |
| Starch + Other | [g/100 g] | 63.1 | 89.2 |
| Aspartic acid | [g/100 g] | [2.59-2.64] | <0.05 |
| Threonine | [g/100 g] | [0.79-0.82] | <0.05 |
| Serine | [g/100 g] | [0.97-1.00] | <0.05 |
| Glutamic acid | [g/100 g] | [3.88-4.19] | <0.05 |
| Proline | [g/100 g] | [0.99-1.02] | <0.05 |
| Glycine | [g/100 g] | [0.99-1.03] | <0.05 |
| Alanine | [g/100 g] | [0.96-1.01] | <0.05 |
| Valine | [g/100 g] | [1.10-1.16] | <0.05 |
| Methionine | [g/100 g] | [0.06-0.1] | <0.05 |
| Isoleucine | [g/100 g] | [1.00-1.06] | <0.05 |
| Leucine | [g/100 g] | [1.67-1.75] | <0.05 |
| Tyrosine | [g/100 g] | [0.73-0.80] | <0.05 |
| Phenylalanine | [g/100 g] | [0.98-1.02] | <0.05 |
| g-aminobutyric | [g/100 g] | <0.05 | <0.05 |
| acid | | | |
| Ornithine | [g/100 g] | <0.05 | <0.05 |
| Lysine | [g/100 g] | [1.41-1.55] | <0.05 |
| Histidine | [g/100 g] | [0.60-0.62] | <0.05 |
| Arginine | [g/100 g] | [2.03-2.12] | <0.05 |
| Taurine | [g/100 g] | <0.05 | <0.05 |
| Hydroxy-Proline | [g/100 g] | <0.05 | <0.05 |
| Tryptophan | [g/100 g] | [0.20-0.24] | <0.05 |
| Hydroxy-lysine | [g/100 g] | <0.05 | <0.05 |
| Total (amino | [g/100 g] | Average 21.53 | <0.05 |
| acids) | | | |
| Vitamin B | [mg/100 g] | | |
| complexes | | | |
| saturated fatty | [g/100 g] | 0.2 | <0.1 |
| acids | | | |
| Mono-unsaturated | [g/100 g] | 0.26 | <0.1 |
| fatty acids | | | |
| Poly-unsaturated | [g/100 g] | 0.85 | <0.1 |
| fatty acids | | | |
| trans fatty acids | [g/100 g] | <0.1 | <0.1 |

Due to the sifting process, a part of the proteins of the legume remains in the starch-containing fraction, which is recognizable by the enclosed amino acid spectrum. In addition, it was found that the subsequent step S4, in particular an enzymatic hydrolysis, also does not significantly change the amino acid spectrum as well as the fat spectrum. This is advantageous because, on the one hand, the amino acids present can be used as nutrients and, on the other hand, the vitamin B complex can also serve as a growth factor for fungi or bacteria. Overall, the hydrolyzed product can thus serve as a basic material for further processing and an addition of further substances can be reduced.

In step S24, the second fraction is now hydrolyzed to produce a starch hydrolysate. Various processes can be used for this. For example, step S24 of the hydrolyzing process is carried out enzymatically. Various sugar-producing enzymes from the group of amylases, such as alpha- and beta-amylase, maltase, dextrinase, saccharase, glycosidase, glucoamylase or pullulanase, are suitable for this purpose. The enzymes used allow the various sugars obtained from starch to be adjusted according to requirements. The enzymes described here can be used individually, but also in combination. Likewise, it is possible to add the enzymes at different times and at different temperatures and pH parameters to obtain a mixture of different sugars in the starch hydrolysate. In a practical step, an amylase is used that has its enzymatic maximum at relatively low temperatures. The use of such enzymes at low temperatures has the advantage that they do not affect any temperature-stable vitamins that may be present, so that they are still present after hydrolysis.

After completion of the hydrolysis in step S24, the enzymes are removed from the starch hydrolysate as required in step S30 or inactivated by addition of chemicals, for example acids or others. Inactivation can also take place via appropriate temperature change, although care would have to be taken here to ensure that this also denatures the residual protein content or also the vitamins if necessary. In the case of inactivation by acid, the added acid can be neutralized again after inactivation by ammonia or other basic compounds. This has the advantage that the starch hydrolysate thus formed contains an additional source of nitrogen, which is useful for further processing steps.

The starch hydrolysate obtained in this way now allows further processing into various sugars or fermentation or further processing into proteins with the aid of a microorganism, in particular a fungal mycelium.

In this respect, FIG. 4 shows an embodiment of such a process in which the starch-containing fraction as a result of the proposed dry extraction process or downstream wet extraction process forms the starting product in step S40. In step S41, enzymatic saccharification is carried out by means of one or more suitable enzymes. These can then be inactivated by acid and the acid neutralized again after an optional filter process in step S42.

The optional filtering process, for example with membrane filtration, retains not only the remaining fibers but also the proteins and possibly also fats, thus separating them from the remaining sugar produced. The protein fractions form a further advantageous side stream, since the membrane filtration or also another suitable measure, can almost completely separate the proteins and/or fats still remaining in the starch-containing fraction. The side stream thus produced is of high concentration and forms, for example, a protein isolate with a protein content greater than 40% by weight, but optionally also greater than 60% by weight or 80% by weight. Due to this multiple structure, almost the entire protein can be extracted from the legume with advantage and further used as concentrate or isolate.

In one embodiment, the starch hydrolysate thus formed in S43 forms the starting product for cultivation with a fungal mycelium and generation of a fungal and legume protein mixture. To this end, an additional nitrogen source is first added in step S44. This can be done, for example, in the form of ammonium, in particular in the form of ammonium sulfate, ammonia or nitrates. Neutralization of the acidic environment in steps S41 or S42 by means of a nitrogenous and basic component is also possible here.

The additional nitrogen source serves some microorganisms such as fungi as a source for the formation of the fungal mycelial protein. Provided that no filtering is carried out, the fungus can, if necessary, also make use of the nitrogen already present from the legume protein, so that an addition of a nitrogen source can be reduced or even completely omitted. In step S45, fungal protein is formed after addition of a suitable fungal mycelium, in particular from the division of the Basidiomycota and/or the Ascomycota.

After the cultivation process is complete, this is dried and ground to produce a fungal protein mixture. Alternatively, the fungal protein mixture can simply be compressed, and then cooled. There are various options for further processing. Depending on whether protein filtering and separation occurred in step S42, the product obtained in S46 forms a mixture of the residual legume protein from the original starch-containing fraction plus the fungal protein or a pure fungal protein in this manner.

It was recognized that the high proportion of B vitamins in the starch-containing fraction is essentially preserved by processing using starch hydrolysate and in subsequent cultivation, so that the fungal and legume protein mixture produced additionally comprises the appropriate proportion of B vitamins. In addition, the presence of minerals from the original starchy fraction makes this mixture particularly nutritious and suitable for processing into vegan foods. Fungi possess different essential amino acids. By utilizing the protein fraction present after hydrolysis, fungi can be used that require the very amino acids found in the legume protein used, such as glutamine and aspartic acid, as essential amino acids. Conversely, by adding the previously separated protein portion after production or even during production of the fungal protein mixture, proportions of individual amino acids can be increased above those present in the original fungal protein mixture.

Furthermore, it was recognized that the protein mixture from fungal mycelia from the division of the Basidiomycota, the Ascomycota or also the Fusarium species comprise a lysine fraction or arginine fraction, which is increased by the use of the legume protein. The starchy fraction from a dry extraction process, in which a legume protein in the range of 5% to 35% by weight is also present, increases the lysine content or arginine content in the final mixture in S46 compared to the lysine or arginine content in the first protein fraction, i.e., the fungal protein mixture. Thus, by appropriate choice of legumes and selection in the formation of the starchy fraction, a balanced mixture of different essential amino acids and thus an improved biovalue can be achieved.

Such a fungal protein mixture is thus characterized in some aspects by the fact that the protein composition or even the proportions of amino acids are partly due to the production with the proposed sift grinding process.

Referring again to FIG. 4, in a further embodiment there is also the possibility of fermentation to obtain end products other than proteins. This further processing can use the hydrolysate formed above as the starting material, with additional nutrients or other growth-promoting components being added as required if these are not present in the hydrolysate or are not present in sufficient quantity.

It should be noted that in some applications, after hydrolysis, the protein portion left over from the sifting process remains in the hydrolysate and is not separated. This is useful when the cost of the nutrients that would otherwise be added and the cost of separating the proteins from the hydrolysate exceed the value of the separated protein portion. Independently of this, depending on further processing, the minerals and trace elements still present can have a promoting effect in addition to proteins. It has been shown in initial trials that a hydrolysate obtained from the starch-rich fraction also leads to a temporary increase in biosynthesis due to the amino acids present. The reason is the valine, leucine, isoleucine, threonine, methionine, phenylalanine and tyrosine present in the hydrolysate, most of which are metabolized in lactic acid bacteria during fermentation. In addition, a strong decrease in serine, asparagine, aspartic acid, glutamic acid, histidine and tryptophan can also be observed.

In step S43, the proteins comprised in the hydrolysate are optionally cleaved and in this way the proportion of free amino acids is increased. This is useful because, depending on the bacterium used, not all of them can cleave the proteins themselves. Sporolactobacillus inulinus, for example, seems to have a lower or more selectively functioning peptide transport, while Lactococcus lactis is better able to process the proteins present by different mechanisms. For this reason, on the one hand, it is appropriate to select a suitable strain depending on the spectrum of amino acids or proteins in the hydrolysate, or to split the proteins. For this purpose, proteases are added in step S43. This step can also be carried out during hydrolysis, provided that the required temperature ranges and/or pH values should match in order to achieve good cleavage.

Another positive effect on lactic acid production is caused by the vitamins present in the hydrolysate, especially those of the B complex. Thus, a lack of thiamine (B1), riboflavin (B2), niacin (B3) and Ca-pantothenate (B5) lead to lower production, as these serve as co-factors for the synthesis of precursors to lactic acid. However, this does not apply to the same extent to pyridoxine (B6), biotin (B7) and folic acid (B9).

In step S45, the bacteria are then fed and stimulated to produce lactic acid. For this purpose, the pH suitable for the production of lactate is adjusted and, by adding a Ca compound, the lactate (for example Ca-lactate) is converted into poorly soluble salt, which precipitates during the synthesis. Calcium carbonate or calcium hydroxide is used as a possible compound here, which also allows pH regulation. The Ca-lactate formed is poorly soluble and therefore precipitates.

Since the biological production of lactate is essentially an equilibrium reaction, the conversion to Ca-lactate continuously removes it, preventing product or pH inhibition. The Ca-lactate can be flushed out and separated from the mixture in step S46. After filtration, and separation of the lactic acid, it is treated and is then available for further processing.

Various aspects of processes for producing lactic acid are given below.

A process for producing lactic acid comprising the steps of:

Providing a starch hydrolysate and/or a protein hydrolysate, in particular from a starch-rich fraction obtained by a sift grinding process with:
a. a sugar content of at least 40% by weight, in particular at least 50% by weight, and in particular greater than 60% by weight, and a legume protein mixture, in particular of fava bean or pea, with a content of more than 5% by weight and less than 35% by weight, in particular with a content of more than 10% by weight and less than 30% by weight, and in particular with a content of between 15% by weight and 25% by weight based on the total dry weight;
   Adding a lactate-producing bacterium or fungus;
   Separation of the lactate;
   Processing of the lactate to lactic acid.

The method according to item 1, wherein the sugar portion comprises at least one of the following sugars in an amount of at least 30% by weight based on the sugar portion:
Glucose;
Fructose;
maltose; and
Sucrose.

The method according to any one of items 1 to 2, wherein the step of adding a lactate-producing bacterium comprises adding at least one bacterial genus from any one of the following:
Lactobacillus;
Leuconostoc;
Pediococcus;
Carnobacterium;
Lactococcus;
Streptococcus;
Enterococcus;
Vagococcus;
Aerococcus;
Alloiococcus;
Oenococcus;
Sporolactobacillus;
Tetragenococcus; and
Weissella.

The method according to any one of the preceding items, wherein the step of adding a lactate-producing bacterium comprises adding at least one kind of
Sporolactobacillus laevolacticus;
Sporolactobacillus inulinus;
Sporolactobacillus putidus;
Lactobacillus lactis;
Lactobacillus delbrueckii or its subtypes;
Lactobacillus coryniformis or its subtypes; and
Leuconostoc mesenteroides
includes.

The method according to any one of the preceding items, wherein a combination of Sporolactobacillus inulinus and Lactococcus lactis is added as a lactate-producing bacterium.

The method according to any one of the preceding items, wherein the step of providing a starch hydrolysate and/or protein hydrolysate comprises adding proteases that at least partially cleave the protein mixture into amino acids prior to adding the bacterium.

The method according to any one of the preceding items, in which the starch hydrolysate and/or protein hydrolysate comprises a free amino acid content in the range of more than 1% by weight and less than 20% by weight, in particular between 5% by weight and 15% by weight and in particular between 4% by weight and 10% by weight based on dry matter.

The method according to any one of the preceding items, wherein calcium carbonate or calcium hydroxide is added in the step of separating the lactate.

The method according to any one of the preceding items, wherein the step of processing the lactate to lactic acid comprises:
Optional dissolving the separated lactate in sulfuric acid and separating of the precipitated sulfate;
Purifying the lactic acid, especially by activated carbon;
Esterification of lactic acid; and
Hydrolyzing the ester.

1. A method of producing a starch hydrolysate comprising the steps of:
Providing at least one type of legume;
Sift grinding the provided at least one type of legume and separating it into a first fraction and a second fraction, wherein
the first fraction comprises a higher protein content than the second fraction; and
in the second fraction, optionally, a proportion of a starch contained in the provided at least one legume species is at least 40% by weight, in particular at least 50% by weight;
Hydrolyzing the second fraction to produce a starch hydrolysate which comprises a protein content in the range of from 5% by weight to 35% by weight, and in particular in the range of from 10% by weight to 30% by weight.

2. The method of claim 1, further comprising:
Wet extracting the first fraction to produce a protein isolate having a legume protein content in the range of from 80% by weight to 97% by weight, and in particular in the range of from 85% by weight to 95% by weight.

3. The method according to claim 1, wherein the step of hydrolyzing comprises filtering, in particular in membrane filters and/or precipitating the hydrolyzed second fraction.

4. The Method according to claim 1, wherein, after the step of hydrolyzing, at least one separation of still present protein portion and/or fat portion takes place by at least one of the following steps:
Filtering, in particular a membrane filtering;
Centrifugating;
Decanting;
Precipitating; and
Combinations of these.

5. The method according to claim 1 further comprising:
Unhulling the legumes prior to the sift grinding step; and/or
Sieving of the second fraction to remove residual material with a particle size greater than 60 μm from the second fraction.

6. The method according to claim 1, wherein in the second fraction, prior to an optional step of separating a still present protein portion and/or fat portion, a protein portion is in at least one of the following ranges:
5% by weight to 35% by weight;
20% by weight to 25% by weight;
22% by weight to 27% by weight;
18% by weight to 23% by weight;
12% by weight to 20% by weight;
15% by weight to 30% by weight;
20% by weight to 30% by weight;
22% by weight to 30% by weight;
24% by weight to 30% by weight.

7. The method according to claim 1, wherein the second fraction prior to hydrolysis comprises a fat content in at least one of the following ranges:
0.8% by weight to 1.6% by weight,
1% by weight to 1.4% by weight;
1.5% by weight to 6.0% by weight;
2.25% by weight to 5.5% by weight;
0.5% by weight to 5.0% by weight;
1.0% by weight to 4.5% by weight;
1.5% by weight to 4% by weight;
1.0% by weight to 3.5% by weight.

8. The method according to claim 1, wherein the hydrolyzed second fraction comprises, prior to an optional step of separating a protein portion and/or fat portion, a fat content of more than 0.5% by weight, and in particular in at least one of the following ranges:
0.8% by weight to 1.6% by weight,
1% by weight to 1.4% by weight;
1.5% by weight to 6.0% by weight;
2.25% by weight to 5.5% by weight;
0.5% by weight to 5.0% by weight;
1.0% by weight to 4.5% by weight;
1.5% by weight to 4% by weight;
1.0% by weight to 3.5% by weight.

9. The method according to claim 1, wherein the hydrolyzed second fraction comprises, prior to an optional step of separating any remaining protein portion, at least one of the following ingredients:
Aspartic acid between 1.5% by weight to 4% by weight, in particular between 2% by weight to 3% by weight and especially between 2.5% by weight to 3.3% by weight, in each case based on the dry mass of the hydrolyzed fraction;
Glutamic acid between 2.7% by weight and 5.5% by weight, in particular between 3.0% by weight and 4.7% by weight and in particular between 3.6% by weight and 4.3% by weight in each case based on the dry mass of the hydrolyzed fraction;
Arginine 1.6% by weight to 2.6% by weight and in particular between 1.9 to 2.2% by weight, in each case based on the dry mass of the hydrolyzed fraction;
Serine or alanine or phenylalanine or proline or glycine, in each case with a proportion between 0.5% by weight and 4.5% by weight in each case based on the dry mass of the hydrolyzed fraction;
Lysine above 4% by weight in each case based on the dry mass of the hydrolyzed fraction.

10. The method according to claim 1, wherein the step of hydrolyzing is performed enzymatically, in particular with at least one enzyme selected from the group consisting of:
alpha-amylase;
beta-amylase;
maltase;
dextrinase;
saccharase;
glycosidase;
glucoamylase; and
pullulanase.

11. The method according to claim 1, wherein the step of hydrolyzing is carried out by means of an acid, wherein, after completion of hydrolyzing, neutralization of the acid is carried out, in particular with ammonia.

12. The method according to claim 1, wherein the starch hydrolysate comprises at least one of the following sugars, each on a dry weight basis:
glucose in the range of 60% by weight to 98% by weight;
fructose in the range of 5% by weight to 30% by weight;
maltose ranging from 5% by weight to 30% by weight; and
sucrose in the range of 2% by weight to 20% by weight.

13. The method according to claim 1, further comprising:
Cultivation of a fungal mycelium from the division of Basidiomycota and/or Ascomycota with the starch hydrolysate as well as an additional nitrogen source;
and at least one of the following steps:
Drying and grinding of the cultivated fungal mycelium to produce a fungal protein mixture;
Cooling the cultivated fungal mycelium;
Wet processing the cultivated fungal mycelium; and
Pasteurizing the cultivated fungal mycelium.

14. The method according to claim 1, further comprising:
Fermenting the starch hydrolysate with a lactic acid bacterium or a fungus to produce lactate, especially Ca-lactate and
Further processing of the lactate formed to lactic acid; or
Fermenting the starch hydrolysate with a microorganism to produce at least one end product substance selected from the group consisting of diols, alcohols, amino acids and vitamins.

15. The method according to claim 14, wherein the step comprises culturing or fermenting:
Adding a nitrogen source, in particular in the form of ammonium, in particular ammonium sulfate, ammonia and/or nitrates; or
Adding at least one amino acid, in particular from the group comprising:
Valine;
Leucine;
Isoleucine;
Threonine;
Methionine;
Phenylalanine and
Tyrosine.

16. The method according to claim 1, wherein providing at least one type of legume comprises providing
Peas;
green beans;
Fava beans;
Chickpeas;
Peanuts;
Lentils;
Soybeans;
Combinations of these.

17. A starch hydrolysate, comprising:
a sugar content of at least 40% by weight, in particular at least 50% by weight, and in particular greater than 60% by weight,
wherein the sugar portion comprises at least one of the following sugars in an amount of at least 10% by weight:
glucose;
fructose;
maltose; and
sucrose;
a legume protein mixture, in particular of fava bean or pea, in a proportion of between 5% by weight and 30% by weight and in particular between 10% by weight and 25% by weight.

18. The starch hydrolysate according to claim 17, wherein the legume protein mixture is in at least one of the following:
5% by weight to 35% by weight;
20% by weight to 25% by weight;
22% by weight to 27% by weight;
18% by weight to 23% by weight;
12% by weight to 20% by weight;
15% by weight to 30% by weight;
20% by weight to 30% by weight;
22% by weight to 30% by weight;
24 weight % to 30 weight %,
in particular based on the dry mass of the starch hydrolysate.

19. The starch hydrolysatre according to claim 18, wherein the starch hydrolysate comprises a fat content from a legume of greater than 0.5% by weight and is in at least one of the following ranges:
0.8% by weight to 1.6% by weight,
1% by weight to 1.4% by weight;
1.5% by weight to 6.0% by weight;
2.25% by weight to 5.5% by weight;
0.5% by weight to 5.0% by weight;
1.0% by weight to 4.5% by weight;
1.5% by weight to 4% by weight;
1.0% by weight to 3.5% by weight.

20. The starch hydrolysate according to claim 17, wherein the starch hydrolysate comprises a proportion of B-complex vitamins in at least one of the following ranges.
1.5 mg to 6 mg based on 100 g total dry weight;
1.8 mg to 5.6 mg based on 100 g total dry weight;
2.0 mg to 5.1 mg based on 100 g total dry weight;
2.2 mg to 4.2 mg based on 100 g total dry weight;
2.5 mg to 4.7 mg based on 100 g total dry weight;
2.8 mg to 4.2 mg based on 100 g total dry weight;
2.8 mg to 5.5 mg based on 100 g total dry weight;
3.2 mg to 6.0 mg based on 100 g total dry weight.

21. The starch hydrolysate according to claim 17, comprising non-proteinaceous portions having a particle size in the range of 30 μm to 70 μm, particularly in the form of dietary fiber and fibrous legume fiber.

22. The starch hydrolysate according to claim 17, further comprising glutamic acid at a weight fraction of from 2.7% to 5.5%, more particularly between 3.0% to 4.7% and more particularly between 3.6% to 4.3% in each case based on the dry matter of the hydrolyzed fraction.

23. The starch hydrolysate according to claim 17, further comprising at least one of the following ingredients:
Aspartic acid with a weight fraction of 1.5% to 4%, in particular between 2% to 3% and especially between 2.5% to 3.3%, in each case based on the dry mass of the hydrolyzed fraction;
Arginine with a weight fraction of 1.6% to 2.6% and in particular between 1.9 to 2.2% by weight, each based on the dry matter of the hydrolyzed fraction;
Serine or Alanine or Phenylalanine or Proline or Glycine, in each case with a proportion by weight of between 0.5% and 4.5%, based on the dry mass of the hydrolyzed fraction;
Lysine with a percentage by weight above 4% in each case based on the dry mass of the hydrolyzed fraction.

24. A fungal protein mixture, comprising:
a first protein portion from a fungus selected from the division of Basidiomycota and/or Ascomycota; and
a second protein portion derived from a hydrolysate used to produce the first protein portion, which is obtained by carrying out a sift grinding process;
wherein the second protein portion increases a lysine portion and/or an arginine portion in the fungal protein mixture relative to the lysine portion and/or arginine portion in the first protein portion.

25. The fungal protein mixture according to claim 24, produced by a process according to the preceding claims.

26. The fungal protein mixture according to claim 24, wherein the second protein portion comprises a legume protein, particularly a pea protein, a fava bean protein, or combinations thereof.

27. The fungal protein mixture according to claim 24, wherein the lysine portion and/or arginine portion in the fungal mycelial protein mixture is in the range of 10% to 30% greater than the lysine portion and/or arginine portion in the first protein portion.

28. A fungal protein mixture according to claim 24, wherein a sugar content is less than 10% by weight and in particular less than 5% by weight.

29. The fungal protein mixture according to claim 24, comprising a level of B vitamins ranging from 0.002% by weight to 0.005% by weight.

30. The fungal protein mixture according to claim 24, which comprises a fat content from a legume of greater than 0.5% by weight and is in at least one of the following ranges:
0.8% by weight to 1.6% by weight,
1% by weight to 1.4% by weight;
1.5% by weight to 6.0% by weight;
1.5% by weight to 3.5% by weight;
2.25% by weight to 5.5% by weight;
0.5% by weight to 5.0% by weight;
1.0% by weight to 4.5% by weight;
1.5% by weight to 4% by weight;
1.0% by weight to 3.5% by weight.
